(19) United States
(12) Patent Application Publication
(10) Pub. No.: US 2015/0335606 A1
(43) Pub. Date: Nov. 26, 2015

(54) Gel Compositions

(71) Applicant: LEO LABORATORIES LIMITED, Dublin (IE)

(72) Inventors: Per-Ola Arvidsson, Ballerup (DK); Edit Farkas, Ballerup (DK); Chinar Sami Saeed, Ballerup (DK); Mikkel Krogh-Madsen, Ballerup (DK)

(21) Appl. No.: 14/652,091

(22) PCT Filed: Dec. 11, 2013
(86) PCT No.: PCT/EP2013/076187

§ 371 Date: Jun. 12, 2015

(30) Foreign Application Priority Data
Dec. 12, 2012 (GB) 1222406.9

(51) Int. Cl. A61K 31/22; A61K 9/00; A61K 9/06; A61K 9/127

(52) U.S. Cl. CPC A61K 31/22; A61K 9/127; A61K 9/0014; A61K 9/06; A61K 9/1272

(57) ABSTRACT

Aqueous topical liposome gel compositions comprising ingenol-3-angelate.

### TECHNICAL FIELD

The invention relates to a topical gel composition comprising an ingenol-3-angelate as a pharmacologically active agent.

### BACKGROUND OF THE INVENTION

PICATO® is an aqueous gel formulation comprising ingenol-3-angelate (2-methyl-2(Z)-butenoic acid (1aR,2S,5R,5aS,6S,8aS,9R,10aR)-5,5a-dihydroxy-4-(hydroxymethyl)-1,1,7,9-tetramethyl-11-oxo-1a,2,5,5a,6,9,10,10a-octahydro-1H-2,8a-methanocyclopenta[a]cyclopropa[e]cyclodecen-6-yl ester, also known as ingenol-3-mebutate or PEP005) at a strength of 0.015% or 0.05%. PICATO® was granted regulatory approval in 2012 by the FDA for the topical treatment of actinic keratosis.

The compound ingenol-3-angelate (PEP005) [Sayed, M. D. et. al.; Experienta, (1980), 36, 1206-1207] can be isolated from various Euphorbia species, and particularly from Euphorbia peplos [Hohmann, J. et. al; Planta Med., (2000), 66, 291-294] and Euphorbia drummondii by extraction followed by chromatography as described in U.S. Pat. No. 7,449,492.

Pharmaceutical formulation of the compound has been described in WO2007/068963, which discloses various gel formulations for the treatment of skin cancer.

Angelic acid and angelic acid esters such as ingenol-3-angelate are prone to isomerisation of the double bond to form the tiglate ester, particularly at basic pH or when subjected to heat [Beeby, P., Tetrahedron Lett. (1977), 38, 3379-3382, Hoskins, W. M., J. Chem. Soc. Perkin Trans. 1, (1977), 538-544, Bohlmann, F. et. al., Chem. Ber. (1970), 103, 561-563].

Furthermore, ingenol-3-acylates are known to be unstable as they rearrange to afford the ingenol-5-acylates and ingenol-20-acylates [Sorg, B. et. al, Z. Naturforsch., (1982), 37B, 748-756].

It is an object of the invention to provide a composition of ingenol-3-angelate which is stable at room temperature for extended periods.

It is a further object of the invention to provide a composition exhibiting favourable penetration characteristics and biological activity.

It is a further object of the invention to provide a composition with reduced skin irritation, favourable cosmetic properties and improved patient compliance.

A crystalline form of ingenol-3-angelate has been described in WO2011/128780. It is also an object of the invention to utilise the properties of the crystalline structure.

### STATEMENT OF INVENTION

The present invention relates to an aqueous topical liposome gel composition comprising ingenol-3-angelate. The ingenol-3-angelate in the composition is usefully present at 0.015% by weight or 0.05% by weight.

The present invention further relates to methods for treating a dermal disease or condition comprising topical administration of a gel of the invention.

### BRIEF DESCRIPTION OF DRAWINGS

FIG. 1 shows the percentage of applied ingenol-3-angelate which penetrates into the viable epidermis and dermis (dark shading) and the percentage of applied ingenol-3-angelate which permeates to receptor fluid (light shading) according to the in vitro diffusion test for composition 14-08A and PICATO® at the same strength of ingenol-3-angelate by weight of the composition.

### DETAILED DESCRIPTION OF THE INVENTION

Human skin, in particular the outer layer, the stratum corneum, provides an effective barrier against penetration of microbial pathogens and toxic chemicals. While this property of skin is generally beneficial, it complicates the dermal administration of pharmaceuticals in that a substantial amount of an active ingredient applied on the skin of a patient suffering from a dermal disease may not penetrate into the viable layers of the skin (the dermis and epidermis) where it exerts its activity. The gel compositions of the present invention may provide advantageous penetration properties whilst reducing the likelihood of skin irritation.

The use of a liposomal system including ingenol-3-angelate may also improve chemical stability, and may result in a satisfactory chemical stability of ingenol-3-angelate permitting the composition to be stored at room temperature (25° C.) for extended periods.

### Gels

Gels are semisolid dosage forms that contain an agent (a gelling agent) to provide stiffness to a solution or a colloidal dispersion. Gels do not flow at low shear stress and generally exhibit plastic flow behaviour. The gel compositions of the present invention could be hydrogels.

Whether or not a system behaves as a gel (i.e. exhibits semisolid characteristics, rather than acting as a liquid or solid, etc.) will depend on the various components used within the system and the relative ratios of the different components. It may also depend on the method by which the components that make up the system are mixed, e.g. the order in which the various components are introduced to each other. It is therefore possible for an agent to act as a gelling agent in one environment but not in another. The ability to test compositions to confirm that they are gels as defined herein is within the knowledge of the skilled person in view of the present disclosure and common general knowledge in the field.

The viscosity of a gel can depend on temperature. At low temperatures (e.g. 2-8° C.) the viscosity can be relatively high, but after applying a gel composition of the invention to the skin it can become less viscous because of the combination of increased temperature and the physical stress while being applied. This shear-thinning characteristic gives a gel which is easily distributed on the skin.

In order to effect formation of a gel, it is necessary to have an agent in the composition which acts as a gelling agent. The amount of the gelling agent (or gelling agents, in embodiments where two or more gelling agents are used) required to form a gel will vary on the components within the particular composition. It is common (although not required) to select two or more components which, when used together in particular amounts, effect formation of a gel. These components would typically include an emulsifier and/or viscosity-increasing ingredient with an aqueous buffer solution.

In some embodiments the gel compositions are colourless. In other embodiments they include a coloured substance, which can make it easy to see where the gel has been applied.

Gel compositions of the invention are usually transparent. In some embodiments, the gel compositions include suspended ingenol-3-angelate solids. In these embodiments, the gel compositions are preferably transparent except for the suspended ingenol-3-angelate solids. In other less preferred embodiments, the gel compositions are turbid in appearance.

The gel compositions of the invention are typically acidic, because it has been found that alkaline conditions (or even insufficiently strong acidic conditions) may contribute to degradation of ingenol-3-angelate within the gel composition. This means that the gel compositions are sufficiently acidic for the ingenol-3-angelate to remain stable at room temperature (25° C.) for extended periods, e.g. for 2 years. Generally, the aqueous compositions of the invention will have a pH of from about 2 to about 6, e.g. pH 2, 2.5, 3, 3.5, 4, or 4.5. Although not required, the compositions of the invention will typically include an aqueous buffer solution. Preferably, the gel compositions have a pH of less than about 4.5, such as less than 4 or less than 3.5

In general, gels are non-invasive and have a localized effect with minimum side effects. Gel compositions of the invention should be suitable for human topical administration. Thus the compositions have the appropriate physical characteristics of topical gels. For instance, the gel compositions have good spreadability, i.e. the gels can readily be spread (e.g. using fingers) after application to the skin to provide a uniform layer. The gel compositions also have excellent extrudability. These properties mean that the gel compositions of the invention are particularly suitable for topical administration. In some embodiments, the gel compositions are applied topically and do not leave a visible residue. The volatile components of the gel compositions may also substantially evaporate to dryness after a certain period of time following topical application. Preferably, the volatile components of the gel composition will evaporate after a therapeutically effective amount of the ingenol-3-angelate has penetrated into the skin (e.g. after about 1 minute, 2 minutes, 5 minutes, 10 minutes, 20 minutes, etc. following topical administration to a subject).

Ingenol-3-angelate

The composition of the invention includes ingenol-3-angelate. Typically, the composition includes ingenol-3-angelate in an amount of from about 0.001% to about 0.5% by weight of the composition. The composition may include ingenol-3-angelate in an amount of about 0.0005%, 0.001%, 0.0025%, 0.005%, 0.01%, 0.015%, 0.025%, 0.05%, 0.075%, 0.1%, 0.125%, 0.15%, 0.2%, 0.25% or 0.5% by weight of the composition. In two particularly preferred embodiments the composition includes ingenol-3-angelate in an amount of 0.05% or 0.015% by weight of the composition.

Ingenol-3-angelate exists in three isoforms: ingenol-3-angelate (isoform b′), ingenol-5-angelate (isoform ‘a’) and ingenol-20-angelate (isoform ‘c’). The compositions of the present invention include ingenol-3-angelate, i.e. isoform ‘b’, which tends to undergo rearrangement to isoform ‘a’ and subsequently to isoform ‘c’. Preferably, the composition includes less than about 1%, and even more preferably less than about 0.5%, of the ‘a’ isoform after a period of 3 months at room temperature (25° C.). Preferably, the composition includes less than about 1%, and even more preferably less than about 0.5%, of the ‘c’ isoform after a period of 3 months at room temperature (25° C.).

In some embodiments, the compositions of the invention may include crystalline ingenol-3-angelate. In certain embodiments, the compositions of the invention include crystalline ingenol-3-angelate in which the crystalline form is not a solvate. In certain embodiments, the compositions of the invention include crystalline ingenol-3-angelate in which the crystalline form is orthorhombic. In certain embodiments, the compositions of the invention include crystalline ingenol-3-angelate in which the crystalline form is characterized by an FTIR-ATR spectrum exhibiting attenuated total reflectance peaks at approximately 3535, 2951, 1712, 1456, 1378, 1246, 1133, 1028 and/or 956 cm⁻¹ (±3 cm⁻¹). In certain embodiments, the compositions of the invention include crystalline ingenol-3-angelate in which the crystalline form has a differential scanning calorimetry curve comprising an event with an onset at about 153±about 5° C. Preferably, when the compositions of the invention include crystalline ingenol-3-angelate, the ingenol-3-angelate has a polymorphic purity of at least about 80%, such as about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the compositions of the invention include amorphous (non-crystalline) ingenol-3-angelate. The compositions can include a mixture of amorphous and crystalline ingenol-3-angelate.

### Liposomes

The compositions of the invention include liposomes. In these compositions, the liposomes serve as reservoirs for the ingenol-3-angelate. The gel compositions of the invention have favorable penetration characteristics, and can provide a localized and controlled system for delivery of ingenol-3-angelate to a subject.

Liposomes are spherical vesicles having a surface membrane composed of one or more lipid bilayers. The liposome membrane can be composed of a single lipid bilayer or several lipid bilayers (multilayered). Liposomes may form spontaneously upon mixing lipids in aqueous media. In the compositions of the invention, ingenol-3-angelate is typically encapsulated within the lipid bilayer or within multilayers of the liposome. Thus the ingenol-3-angelate is inside the liposomes, typically within the lipid bilayer.

The term “aqueous” means that the content of free water in the composition is greater than or equal to about 2% by weight, preferably more than about 5% by weight of the composition, e.g. more than about 10%, 20%, 30%, 40%, 50%, 60%, 70%, 80% or 90% by weight of the composition.

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x+10%.

Liposome gels are known in the art. For example, liposome gels comprising lidocaine hydrochloride, an anaesthetic agent, have been produced [Glavas-Dodov, M., et al., Bulletin of the Chemists and Technologists of Macedonia, (2005), 24, 59-65].

The liposomes are formed from one or more naturally occurring or synthetic lipid compounds, or a mixture thereof. Suitable lipids include detergents, surfactants, soaps, phospholipids, ether lipids, glycoglycerolipids, etc.

More specific examples of suitable lipids include unsaturated fatty acids (e.g. myristoleic, palmitoleic, elaidic, petroselinic, oleic, vaccenic, gondoic, erucic, nervonic, linoleic, gamma-linolenic, linolenic, arachidonic, eicosapentaenoic, docosahexaenoic acids, etc.), the corresponding fatty acid derivatives (e.g. amides, esters, etc.), the corresponding sulfonic acids, the corresponding sulfonic acid derivatives (e.g. sulfonamides, sulfonate esters, etc.), the corresponding fatty alcohols, etc.

In some embodiments the lipid includes one or more unsaturated acyl moieties. In other embodiments the lipid is a phospholipid, such as natural or synthetic phospholipids, saturated or unsaturated phospholipids, or phospholipid-like molecules. The phospholipid typically includes one or more saturated or unsaturated acyl moieties. In some embodiments the unsaturated acyl moiety is C₁₂-C₂₄ alkenyl. In some embodiments the saturated acyl moiety is C₁₂-C₂₄ alkyl.

Particularly suitable phospholipids include e.g. soybean lecithin, egg lecithin, lecithin, lysolecithin, phosphatidylserine, phosphatidylethanolamine, phosphatidylcholine and phosphatidylinositol, phosphatidylglycerol, phosphatidylacid, etc. In some embodiments, the phospholipids are mixed with a sterol such as cholesterol, which can stabilize the phospholipid system. In some embodiments, the lipid is chemically or physically modified. Modifications alter the properties of the lipid and of the resulting liposome vesicles. Methods of modifying lipids are known in the art of liposomal formulations. In preferred embodiments, the gel composition includes a phospholipid such as those available under the trade names Phospholipon® 90G, Phospholipon® 19H, NanoSolve® or Lipoid SPC®. In some embodiments the phosphoipids are mixed with alcohol. Ethosomes are composed mainly of phospholipids, (phosphatidylcholine, phosphatidylserine, phosphatitidic acid), high concentration of ethanol and water. The high concentration of ethanol makes the ethosomes unique, as ethanol is known for its disturbance of skin lipid bilayer organization; therefore, when integrated into a vesicle membrane, it gives that vesicle the ability to penetrate the stratum corneum.

Additional non-phosphorous-containing lipids suitable for use in the compositions of the present invention include stearylamines, fatty acids, fatty acid amides, fatty alcohol ethers, fatty alcohols, fatty alcohol phosphates etc.

Suitable non-phosphorous-containing lipids include but are not limited to C₆-C₂₂ fatty acids and alcohols, such as stearyl alcohol, capric acid, caprylic acid, lauric acid, myristic acid, stearic acid, oleic acid, linoleic acid, linolenic acid, arachnidoic acid, behenic acid, and their corresponding pharmaceutically acceptable salts. In some embodiments the non-phosphorous-containing lipids include surfactants such as sodium dioctyl sulfosuccinate, sodium lauryl sulfate, amide esters, (e.g. lauric acid diethanolamide, sodium lauryl sarcosinate, lauroyl carnitine, palmitoyl carnitine and myristoyl carnitine), esters with hydroxy-acids (e.g. sodium stearoyl lactylate), sugar esters (e.g. lauryl lactate, glucose monocaprylate, diglucose monocaprylate, sucrose laurate, sorbitan monolaurate, sorbitan monopalmitate, sorbitan monooleate sorbitansesquioleate, sorbitan monostearate and sorbitan tristearate), lower alcohol fatty acid esters (e.g. ethyl oleate, isopropyl myristate and isopropyl palmitate) esters with propylene glycol (e.g. propylene glycol monolaurate, propylene glycol ricinoleate, propylene glycol monooleate, propylene glycol monocaprylate, propylene glycol dicaprylate/dicaprate and propylene glycol dioctanoate), esters with glycerol (e.g. glyceryl monooleate, glyceryl ricinoleate, glyceryl laurate, glyceryl dilaurate, glyceryl dioleate, glycerol monolinoleate, glyceryl mono/dioleate, glyceryl caprylate/caprate, caprylic acid mono/diglycerides, mono- and diacetylated monoglycerides, triglycerides (e.g. corn oil, almond oil, soybean oil, coconut oil, castor oil, hydrogenated castor oil, hydrogenated coconut oil, Pureco 100, Hydrokote AP5, Captex 300, 350, Miglyol 812, Miglyol 818 and Gelucire 33/01), mixtures of propylene glycol esters and glycerol esters (e.g. mixture of oleic acid esters of propylene glycol and glycerol and polyglycerized fatty acids such as polyglyceryl oleate), polyglyceryl-2 dioleate, polyglyceryl-10 trioleate, polyglyceryl-10 laurate, polyglyceryl-10 oleate, and polyglyceryl-10 mono, dioleate (Caprol® PEG 860)). Other suitable non-phosphorous-containing lipids include polyethoxylated fatty acids, (e.g. PEG-8 laurate, PEG-8 oleate, PEG-8 stearate, PEG-9 oleate, PEG-10 laurate, PEG-5 oleate, PEG-10 oleate, PEG-12 laurate, PEG-12 oleate, PEG-15 oleate, PEG-20 laurate and PEG-20 oleate) PEG-fatty acid diesters (e.g. PEG-20 dilaurate, PEG-20 dioleate, PEG-20 distearate, PEG-32 dilaurate and PEG-32 dioleate) PEG-fatty acid mono- and di-ester mixtures, polyethylene glycol glycerol fatty acid esters (e.g. PEGylated glycerol 12-acyloxy-stearate, PEG-20 glyceryl laurate, PEG-30 glyceryl laurate, PEG-40 glyceryl laurate, PEG-20 glyceryl oleate and PEG-30 glyceryl oleate) and alcohol-oil transesterification products (e.g. polyoxyl 40 castor oil, polyoxyl 35 castor oil, PEG-25 trioleate, PEG-60 corn glycerides, PEG-60 almond oil, PEG-40 palm kernel oil, PEG-50 castor oil, PEG-50 hydrogenated castor oil, PEG-60 hydrogenated castor oil, PEG-8 caprylic/capric glycerides, lauroyl macrogol-32 glycerides, linoleoyl macrogolglycerides), stearoyl macrogol-32 glycerides, and PEG-6 caprylic/capric glycerides). The lipid may be Imwitor® 375 (glyceryl citrate/lactate/linoleate/oleate), Polyglyceryl-3 Polyricinoleate or ethyl oleate.

In certain embodiments of the invention the composition includes a hydrophilic non-ionic surfactant combined with a lipid or a lipophilic non-ionic surfactant. Participation of non-ionic surfactants instead of phospholipids in the lipid bilayer results in niosomes

The term “hydrophilic surfactant” means an oil-in-water surfactant with a hydrophilic-lipophilic balance (HLB) value of 9-18, and “lipophilic surfactant” means a water-in-oil surfactant with an HLB value of 1.5-9. By way of an example, polysorbate 80 has an HLB value of 15 and is therefore a hydrophilic surfactant, whereas sorbitan trioleate has an HLB value of 1.8 and is therefore a lipophilic surfactant. The HLB of mixed surfactants is calculated according to their relative weightings (by volume) e.g. a 1:1 mixture by volume of polysorbate 80 and sorbitan trioleate has a HLB of 8.4.

In one embodiment, the composition includes a hydrophilic non-ionic surfactant in an amount of from about 1% to about 40% by weight of the composition, optionally from about 2% to about 15% by weight of the composition. Preferably, the composition includes a hydrophilic non-ionic surfactant in an amount of from about 2% to about 10% by weight of the composition, such as from about 2.5% to about 5% by weight of the composition.

The hydrophilic non-ionic surfactant may be a polyethylene glycol ester of a vegetable oil containing at least 20 moles of ethylene oxide groups/mole of glyceride. Suitable polyethylene glycol esters are typically selected from polyoxyethylene castor oil derivatives (e.g. PEG 20, 30, 35, 38, 40, 50 and 60 castor oil or PEG 20, 25, 30, 40, 45, 50, 60 and 80 hydrogenated castor oil), PEG 20 and 60 corn glycerides, PEG 20 and 60 almond glycerides, PEG 40 palm kernel oil, sodium laurate sulfate, sucrose esters (e.g. sucrose stearate, sucrose distearate, sucrose cocoate or sucrose monolaurate), PEG cocoglyceride, PEG 8 caprylocaprate, polyglyceryl esters and linolenamide DEA. In a preferred embodiment, the hydrophilic non-ionic surfactant is sucrose distearate, such as that available under the trade name Sisterna® SP30.

In certain embodiments, the hydrophilic non-ionic surfactant may be a mixture of acrylamide acryloyldimethyl taurate copolymer, isohexadecane and polysorbate 80, such as that available under the trade name SEPINEO™ P600. The hydrophilic non-ionic surfactant may be an alkylpolyglucoside, such as that available under the trade name SEPINEO™ SE68.

In one embodiment, the composition includes a lipophilic non-ionic surfactant in an amount of from about 0.1% to about 5% by weight of the composition. In other embodiments, the lipophilic non-ionic surfactant may be present in an amount of from about 0.1% to about 40% by weight of the composition. Surfactants are generally irritants, and so it is preferred to use only low levels of certain surfactants. However, some lipophilic non-ionic surfactants, such as monoglyceride esters, are less irritative and so can be present in higher amounts without causing significant levels of skin irritation.

The lipophilic non-ionic surfactant may be selected from monoglyceride esters of C₆₋₂₂ fatty acids (e.g. glyceryl monocaprylate, glyceryl monocaprate, glyceryl monostearate, glyceryl monobehenate), diglyceride esters of C₆₋₂₂ fatty acids (e.g. glyceryl dilaurate), mono- and diglyceride esters of C₆₋₂₂ fatty acids (e.g. caprylic/capric mono- and diglyceride, glyceryl mono- and diricinoleate), propylene glycol esters of C₆₋₂₂ fatty esters (e.g. propylene glycol monocaprylate, propylene glycol monolaurate), dialkylene glycol monoalkyl ethers (e.g. diethylene glycol monoethyl ether), polyglyceryl C₆₋₂₂ fatty acid esters (e.g. polyglyceryl-3-diisostearate), polyethylene glycol esters of a triglyceride/vegetable oil containing 4 to 8 moles of ethylene oxide groups/mole of glyceride (e.g. PEG-6 corn oil, PEG-6 almond oil, PEG-6 apricot kernel oil, PEG-6 olive oil, PEG-6 peanut oil, PEG-6 palm kernel oil, hydrogenated palm kernel oil, PEG-6 triolein, PEG-8 corn oil), polysorbates (e.g. polysorbate 20, polysorbate 40, polysorbate 60, polysorbate 80). In a preferred embodiment, the lipophilic non-ionic surfactant is a sorbitan ester, such as that available under the trade name Span® 120. In a preferred embodiment, the lipophilic non-ionic surfactant is an oleoyl macrogol-6 glyceride, such as that available under the trade name Labrafil® M1944 or a lauroyl polyoxyl-6 glyceride, such as that available under the trade name Labrafil® M2130.

The compositions of the invention may include a mixture of different lipids, e.g. one or more phospholipids and one or more non-phosphorous-containing lipids within the same composition.

The one or more lipids is typically present in the composition in a combined amount of from about 0.1% to about 98% by weight of the composition, such as 0.5% to about 98% by weight of the composition, e.g. from about 1% to about 98% by weight of the composition, from about 0.1% to about 60% by weight of the composition, from about 2.5% to about 75% by weight of the composition, from about 0.1% to about 50% by weight of the composition, from about 5% to about 50% by weight of the composition, from about 0.1% to about 40% by weight of the composition, from about 5% to about 30% by weight of the composition, or from about 5% to about 40% by weight of the composition. The lipid may be present in the composition in an amount of from about 10%, about 15%, about 20%, about 25% or about 30% to about 40% by weight of the composition. Typically, one or more lipids is present in an amount of about 10% by weight of the composition.

### Buffers

The aqueous compositions of the invention typically include an aqueous buffer solution. The use of buffer solutions means that fluctuations in pH can be minimised and thus the pH can be kept more readily within the desired pH range, such as at a pH of less than about 6. This reduces the tendency of the ingenol-3-angelate to degrade to form the tiglate ester, which typically occurs in more basic conditions.

Suitable buffer solutions that can be used in the compositions of the invention include e.g. citrate buffer, phosphate buffer, acetate buffer and citrate-phosphate buffer. A citrate buffer is particularly preferred. If a buffer solution is used in the compositions, the pH of the composition will depend on the amount of buffer and the pH of the buffer used. Typically, the compositions of the invention comprise from about 2.5% to about 90% buffer solution by weight of the composition, e.g. 2.5%, 5%, 10%, 20%, 30%, 40%, 50%, 60%, 70%, 80% or 90% buffer solution by weight of the composition. The pH of the buffer will typically be between about 2 to about 4.5, e.g. pH 2, 2.5, 3, 3.5, 4, or 4.5. A buffer having a pH of from about 2.5 to about 3.5 is particularly preferred for ingenol-3-angelate because this pH range may permit the composition to be stored at room temperature (25° C.) for extended periods. For instance, in preferred embodiments the pH of the buffer is 2.0, 2.1, 2.2, 2.3, 2.4, 2.5, 2.6, 2.7, 2.8, 2.9 or 3.0. Most preferably, the buffer having a pH of from about 2.5 to about 3.5 is particularly preferred for ingenol-3-. For example, a citrate buffer can be made by mixing sodium citrate with water. Methods of making buffers of the type disclosed herein are well known to the skilled person.

### Emulsifiers

In some embodiments, the composition may include an emulsifier. The sulsifier can function as a gelling agent, such that e.g. formulation of a gel may be effected when an emulsifier is added to a mixture of ingenol-3-angelate and an aqueous buffer solution.

The composition may include one or more emulsifiers selected from e.g. polyacrylates, polycarbophils, poloxamers, hyaluronic acid, xanthan, natural polysaccharides, chitosan and cellulose-derivatives. Suitable cellulose-derivative viscosity enhancers include hydroxyalkyl cellulose polymers (e.g. hydroxymethyl cellulose, hydroxyethyl cellulose, hydroxypropyl cellulose (hypromellose) and hydroxypropylmethyl cellulose), carboxymethyl cellulose, methylhydroxyethyl cellulose and methylcellulose, carbomer (e.g. Carbopol®), and carrageenans. In certain preferred embodiments, the emulsifier is hydroxyethyl cellulose, such as that available under the trade name Natrosol® (e.g. Natrosol® 250 HX, Natrosol® PLUS CS, Grade 300 etc.) and METHOCEL®. In certain preferred embodiments, the emulsifier is hydroxypropyl cellulose, such as that available under the trade name Klucel® and METHOCEL®. Typically, the emulsifier is present in an amount of from about 1% to about 20% by weight of the composition, such as about 1%, 3%, 5%, 10%, 15% or 20% by weight of the composition. The composition may include more than one viscosity enhancers, such as two or three viscosity enhancers.

### Non-Aqueous Carrier

The compositions of the invention may include a pharmaceutically acceptable non-aqueous carrier. The non-aqueous carrier may function as a vehicle for the liposomes, such that the liposomes containing the ingenol-3-angelate are dispersed throughout the carrier. The compositions of the invention can include more than one non-aqueous carrier, e.g. two, three, four or five non-aqueous carriers. The one or more non-aqueous carriers are typically present in the compositions in a combined amount of from about 1% to about 40% by weight of the composition, e.g. about 10% by weight of the composition.

In some embodiment, the non-aqueous carrier can act as an occlusive agent, e.g. it can form a layer on the surface of the skin on application of the composition. This layer can form a hydration barrier sufficient to result in reduction of trans-epidermal water loss, thereby improving in skin hydration.

The non-aqueous carrier may be selected from a mineral oil (e.g. liquid paraffin) or a hydrocarbon or mixture of hydrocarbons with chain lengths ranging from C₅ to C₆₀. The non-aqueous carrier may be petrolatum or white soft paraffin. Such a mixture is usually composed of hydrocarbons of different chain lengths peaking at about C₄₀₋₄₄. The non-aqueous carrier may comprise a mixture of petrolatum and liquid paraffin. Such a mixture may consist of hydrocarbons of different chain lengths peaking at C₂₈₋₄₀.

While petrolatum provides occlusion of the treated skin surface, reducing transdermal loss of water and potentiating the therapeutic effect of the active ingredient in the composition, it tends to have a greasy and/or tacky feel which persists for quite some time after application, and it is not easily spreadable on the skin. It may therefore be preferred to employ paraffins consisting of hydrocarbons of a somewhat lower chain length, e.g. paraffins comprising hydrocarbons with chain lengths peaking at C₁₄₋₁₆, C₁₈₋₂₂, C₂₀₋₂₂, C₂₀₋₂₆ or mixtures thereof. The hydrocarbon composition of the paraffins can be determined using gas chromatography. It has been found that paraffins comprising hydrocarbons with chain lengths peaking at C₁₄₋₁₆, C₁₈₋₂₂, C₂₀₋₂₂, C₂₀₋₂₆ or mixtures thereof are more cosmetically acceptable because they are less tacky and/or greasy on application and more easily spreadable. They are therefore expected to result in improved patient compliance. Suitable paraffins of this type, which are generally termed petrolatum jelly, are manufactured by Sonneborn and marketed under the trade name Sonnecone. In preferred embodiments of the invention the non-aqueous carrier is selected from Sonnecone CM, Sonnecone DM1, Sonnecone DM2 and Sonnecone HV. These paraffins are further disclosed and characterized in WO 2008/141078 which is incorporated herein by reference.

In some embodiments the non-aqueous carrier is an iso-paraffin, e.g. isohexadecane or squalane.

The non-aqueous carrier may also be a silicone. In some embodiments, the silicone is cyclic, e.g. cyclomethicone. In other embodiments, the silicone can be linear. In other embodiments, the silicone may be branched. Silicones such as cyclomethicone and dimethicone may be used to reduce the viscosity of the composition, for example in embodiments which also include a silicone of higher viscosity.

In some embodiments, the silicone is a solid mixture of stearoxytrimethylsilane and stearyl alcohol, such as that available under the trade name Dow Corning® Silky Wax 10. In some embodiments, the silicone is a mixture of high molecular weight silicone elastomer (12%) and decamethylcyclopentasiloxane (i.e. a cyclopentasiloxane and dimethicone crosspolymer), such as that available under the trade name Dow Corning® ST-Elastomer 10. In other embodiments, the silicone is comprised of a volatile polydimethylcyclosiloxane composed mainly of decamethylcyclopentasiloxane, such as that available under the trade name Dow Corning® ST cyclomethicone (5-NF). Dow Corning® ST cyclomethicone (5-NF) is particularly useful when the composition comprises a further silicone of higher viscosity, such as Dow Corning® ST-Elastomer 10. In some embodiments, the silicone comprises a cyclopentasiloxane and polyoxyethylene/polyoxypropylene dimethicone, such as that available under the trade name Dow Corning® BY 11-030. In some embodiments the composition includes more than one silicone non-aqueous carrier, e.g. two or three silicones.

The non-aqueous carrier may also be an oily solvent. In one embodiment, the oily solvent may be acylglyceride, where C₆₋₂₂ acylglyceride means a triglyceride or a mixture of mono- and a C₆₋₂₂ diglycerides or mono-, di- and triglycerides of C₆₋₂₂ fatty acids, where C₆₋₂₂ acylglyceride means a triglyceride or a mixture of mono- and diglycerides or mono-, di- and triglycerides of C₆₋₂₂ fatty acids. For example, the oily solvent may be a vegetable oil (e.g. sesame oil, sunflower oil, palm kernel oil, corn oil, safflower oil, olive oil, avocado oil, jojoba oil, almond oil, canola oil, coconut oil, cottonseed oil, peanut oil, soybean oil, wheat germ oil, grape kernel oil etc.), or highly purified vegetable oil (e.g. medium chain triglycerides, long chain triglycerides, castor oil, caprylic/capric mono- and diglycerides, caprylic/capric mono-, di- and triglycerides, etc.). Medium chain triglycerides are triglyceride esters of fatty acids with a chain length of 6-12 carbon atoms. A preferred medium chain triglyceride is a mixture of caprylic (C₈) and capric (C₁₀) triglycerides, e.g. available under the trade name Miglyol 812. Other particularly suitable oily solvents include fatty acid glycerol polyglycol esters, e.g. available under the trade name Cremophor RH40. Particularly suitable caprylic/capric glycerides are available under the trade name Akoline MCM.

In another embodiment, the oily solvent may be a synthetic oil such as a fatty alcohol ester of a C₁₀₋₁₈ alkanoic acid (e.g. isopropyl myristate, isopropyl palmitate, isopropyl linoleate, isopropyl monooleate and isostearyl isostearate etc.), such as that available under the trade name Polawax®.

In another embodiment, the oily solvent may be a polyoxypropylene fatty alkyl ether (e.g. polyoxypropylene-15-stearyl ether, polyoxypropylene-11-stearyl ether, polyoxypropylene-14-butyl ether, polyoxypropylene-10-cetyl ether or polyoxypropylene-3-myristyl ether etc.). The oily solvent may be a stearyl ether such as that available under the trade name Arlamol® E.

The oily solvent may be an alkyl or dialkyl ester such as ethyl oleate, diisopropyl adipate or cetearyl octanoate. The oily solvent may also be a mono- or diglyceride such as glyceryl monooleate, or a fatty alcohol such as oleyl alcohol.

In some embodiments the composition may include a mixture of two oily solvents, or optionally three oily solvents.

### Viscosity-Increasing Ingredient

The gel compositions may include a viscosity-increasing ingredient. For example, when the composition comprises a substantial amount of aqueous buffer solution (e.g. above about 60% by weight of the composition), it may be necessary to add one or more viscosity-increasing ingredients (e.g. in an amount of e.g. about 5% by weight of the composition) in order to form a gel. The viscosity-increasing ingredient may therefore function as the gelling agent. However, there may be no requirement for the additional of a viscosity-increasing ingredient if other components in the composition are capable of acting as a gelling agent.

The viscosity-increasing ingredient can be a wax. The wax may be a mineral wax composed of a mixture of high molecular weight hydrocarbons (e.g. saturated C₃₅₋₇₀ alkanes), such as microcrystalline wax. Alternatively, the wax may be a vegetable or animal wax (e.g. esters of C₁₄₋₃₂ fatty acids and C₁₄₋₃₂ fatty alcohols), such as beeswax or hydrogenated castor oil. In some preferred embodiments the viscosity-increasing ingredient is an inorganic substance such as fumed silica (e.g. available under the trade name Aerosil®, such as Aerosil® 200P, which is a high purity amorphous anhydrous colloidal silicon dioxide). The viscosity-increasing ingredient may also be selected from magnesium stearate, aluminium stearate, a sterol such as cholesterol, a long-chain saturated fatty alcohol such as cetostearyl alcohol. In some preferred embodiments the viscosity-increasing ingredient is a silicone rubber or wax, such as Dow Corning® ST-Elastomer 10 or Dow Corning® Silky Wax 10. Dow Corning® ST-Elastomer 10 and/or Aerosil® are particularly preferred. The composition may include more than one viscosity-increasing ingredient, such as two or three viscosity-increasing ingredients. The viscosity-increasing ingredient may be a mixture of acrylamide acryloyldimethyl taurate copolymer, isohexadecane and polysorbate 80, such as that available under the trade name SEPINEO™ P600. The viscosity-increasing ingredient may be an alkylpolyglucoside, such as that available under the trade name SEPINEO™ SE68.

The amount of viscosity-increasing ingredient may vary (according to the viscosifying power of the ingredient), but the composition may include from about 0.5% to about 40% viscosity-increasing ingredient by weight of the composition. When the viscosity-increasing ingredient is microcrystalline wax it is typically present in an amount of from about 0.5% to about 10% by weight of the composition. Where the viscosity-increasing ingredient is SEPINEO™ P600, it is typically included in an amount of from about 1% to about 10% by weight of the composition, e.g. about 10% by weight of the composition. Where the viscosity-increasing ingredient is SEPINEO™ SE68, it is typically included in an amount of from about 2% to about 30% by weight of the composition, e.g. about 5% by weight of the composition. Where the viscosity-increasing ingredient is Dow Corning® ST-Elastomer 10 and/or Aerosil®, it is typically included in an amount of from about 1% to about 10% by weight of the composition, e.g. 1%, 2%, 5% or 10% by weight of the composition.

### Co-Solvents

In some embodiments, the composition may include a co-solvent selected from the group consisting of lower alcohols, such as ethanol, n-propanol, isopropanol, n-butanol, 2-butanol and benzyl alcohol, and diols such as propylene glycol. This may be preferred where dispersion of the ingenol-3-angelate and/or liposomes is problematic. These co-solvents may also act as a penetration enhancer aiding the penetration of the ingenol-3-angelate into the skin. Addition of a co-solvent may result in an improved physical stability of the composition. The composition may include more than one co-solvent, e.g. two or three co-solvents. For example, the composition may include benzyl alcohol and isopropanol.

The co-solvent may be present in an amount of from present in an amount of from about 0.5% to about 40% by weight of the composition, such as from about 5% to about 30%, e.g. about 10%, about 15%, about 20%, about 25%, or about 30% by weight of the composition.

### Penetration Enhancers

The liposomal compositions of the invention typically have excellent penetration characteristics. However, if it is desirable to further increase penetration, it may be useful to include one or more penetration enhancers. Typical penetration enhancers include propylene carbonate, transcutol, pyrrolidones such as N-methylpyrrolidone or N-hydroxyalkylpyrrolidone, azone, menthol, eucalyptol, nicotinamide, glycerol, mono-di- or polyglycols, ethylacetate or Eugenol. A particularly preferred penetration enhancer is α-tocopherol.

In one embodiment, the composition includes a penetration enhancer in an amount of from about 0.01% to about 20% by weight of the composition, such as from about 0.1% to about 15%, e.g. about 0.1%, about 0.5%, about 1%, about 1.5%, about 2%, about 2.5%, about 3%, or about 5% by weight of the composition.

In one embodiment, the co-solvent (which may function as a penetration enhancer) and a further penetration enhancer are both present in a combined amount of from about 0.01% to about 20% by weight of the composition, such as from about 0.1% to about 15%, e.g. about 0.1%, about 0.5%, about 1%, about 1.5%, about 2%, about 2.5%, about 3%, or about 5% by weight of the composition.

### Acidifying Compounds

The composition of the invention may include an acidifying compound, for example where the stability of the gel composition would otherwise be unsatisfactory. An acidifying compound is a compound capable of providing a net overall acidic environment to the composition which means that the gel compositions are sufficiently acidic for the ingenol-3-angelate to remain stable at room temperature (25° C.) for extended periods, e.g. for 2 years. Generally, the acidifying compounds described herein are compounds which give a pH to the composition of less than about 6, such as less than 4 or less than 3.5.

The composition may include more than one acidifying compounds, for instance it may include two or three acidifying compounds. The acidifying compound may be present in an amount of from about which may be included in the composition in an amount of from about 0.5% to about 10% by weight of the composition, such as from about 5% to about 9% by weight of the composition. In some embodiments, the one or more non-aqueous carriers or the aqueous buffer solution may act as an acidifying compound. The acidifying compound may be fumed silica, which may be included in the composition in an amount of from about 3% to about 10% by weight of the composition, such as from about 5% to about 9% by weight of the composition. Alternatively, the acidifying compound may be a fatty acid such as oleic acid, lactic acid, linoleic acid, stearic acid, lauric acid, palmitic acid, capric acid, caprylic acid, pelargonic acid, adipic acid, sebacic acid or enanthic acid. The fatty acid is typically present in an amount of from about 0.5% to about 5% by weight of the composition.

### Keratinolytic Agents

In some embodiments, the composition includes a keratinolytic agent, such as an α-hydroxy acid or β-hydroxy acid. The use of a keratinolytic agent may improve penetration of the active substance, meaning that compositions comprising a keratinolytic agent are particularly useful for treating hyperkeratotic actinic keratosis.

Suitable keratinolytic agents for use in the compositions of the invention include retinoids, adapalene, tars, shale oil, allantoin, aluminium oxide, azelaic acid, benzoyl peroxide, lactic acid, salicylic acid, alcali and alkali earth sulfide, monochloroacetic acid, urea, and resorcin. Particular retinoids that may be suitable include retinol, retinaldehyde, retinoic acid, isotretinoin, adapalinen and tazarotene. Further keratinolytic agents include ammonium glycolate, ammonium lactate, betaine salicylate, calcium lactate, calcium thioglycolate, glycolic acid, lactic acid, phenol, potassium lactate and sodium lactate.

In one embodiment, the composition includes an α-hydroxy acid selected from glycolic acid, lactic acid, mandelic acid, malic acid, citric acid and tartaric acid. In another embodiment, the composition includes a β-hydroxy acid such as salicylic acid. Preferably, the keratinolytic agent is salicylic acid.

The keratinolytic agent (e.g. α-hydroxy acid or β-hydroxy acid) may be present in an amount of from about 0.1% to about 20% by weight of the composition, e.g. about 0.5%, 1.0%, 2.5%, 5.0%, 7.5%, 10%, 15% or 20% by weight of the composition. Preferably, the composition includes salicylic acid in an amount of from about 0.1% to about 20% by weight of the composition, e.g. about 0.5%, 1.0%, 2.5%, 5.0%, 7.5%, 10%, 15% or 20% by weight of the composition.

### Preferred Embodiments

In an embodiment the invention comprises ingenol-3-angelate, lipid and alcohol. In embodiments the compositions additionally comprises buffer. In embodiments the composition additionally comprises hydroxyethylcellulose or Sepineo P600.

### Processes

The compositions of the invention can be made by mixing together ingenol-3-angelate and one or more naturally occurring or synthetic lipid compounds which are capable of forming liposomes. A solvent may be added, if necessary, to dissolve the ingenol-3-angelate. This solvent may be the same as the co-solvent as defined herein, and may in some embodiments function as a co-solvent in the final composition. Alternatively, the solvent can be removed after formation of the liposome composition, for instance using vacuum evaporation, to yield a dry lipid film. If the solvent is intended to be removed, it is preferable to use an alcohol, halogenated organic solvent, ether or ketone which has a low vapour pressure. Diethyl ether, acetone, dichloromethane, methanol, ethanol and propylene glycol are particularly suitable. If the solvent is intended to remain in the composition as a co-solvent, ethanol, isopropanol and propylene glycol are particularly suitable.

The mixture (either a solution or a dried lipid film) is typically mixed with an aqueous buffer solution, followed by homogenization by means of a dispersion process. It is possible to make the compositions of the invention without a drying step by subjecting the ingenol-3-angelate, the one or more naturally occurring or synthetic lipid compounds and the aqueous buffer solution to a dispersion process directly.

In some embodiments, the gel compositions can be made by dissolving phospholipid in an alcohol, followed by addition of ingenol-3-angelate under stirring. Buffer solution may then be added, followed by homogenization. A gelling agent can be separately mixed with further buffer solution, and this mixture may be added to the homogenized phospholipid, alcohol, ingenol-3-angelate and buffer solution mixture. Finally, benzyl alcohol may be added.

In other embodiments, phospholipid may be mixed with buffer solution under mixing, followed by addition of ingenol-3-angelate. Benzyl alcohol can then be added. A gelling agent can be separately mixed with further buffer solution, and this mixture may be added to the phospholipid, ingenol-3-angelate, buffer solution and benzyl alcohol mixture. The mixture may then be stirred until homogeneous.

### Stability of the Compositions

The inventors have found that compositions of the invention exhibit very favorable stability properties.

In some embodiments, the composition is chemically stable, where chemically stable (or chemical stability) means that less than 10% of the ingenol-3-angelate degrades when the gel is stored for 2 years at 25° C. In some preferred embodiments, less than 6% of the ingenol-3-angelate degrades over a storage period of 2 years. An approximation of chemical stability can be obtained by subjecting the composition to stability studies at 25° C. for 6 months: if less than about 2.5% of the ingenol-3-angelate has degraded after 6 months at 25° C. then a shelf-life of 2 years at room temperature is expected, i.e. less than 10% of the ingenol-3-angelate will be expected to degrade over a storage period of 2 years at 25° C. An approximation of chemical stability at room temperature can also be obtained by subjecting the composition to accelerated stability studies at 40° C. for 3 months. If less than about 2.5% of the substance, e.g. ingenol-3-angelate, has degraded after 3 months at 40° C., a shelf-life of 2 years at room temperature is considered to be feasible. These studies are carried out according to ICH Humidity Guidelines, at conditions of 25° C.±2°, 60% RH±5% and/or 40° C.±2°, 75% RH±5%, in hermetically Preferred chemically stable gels include, after storage for 2 years at 25° C., less than 5% by weight of total ingenanes in the composition are ‘A’ and/or ‘B’. Thus, if the total amount of ‘A’ and ‘B’ exceeds 5% by weight of the total ingenanes, the gel's shelf-life is not ideal. An approximation of the amount of ‘A’ and/or ‘B’ in the embodiments can be carried out in the same manner as described for ingenol-3-angelate above.

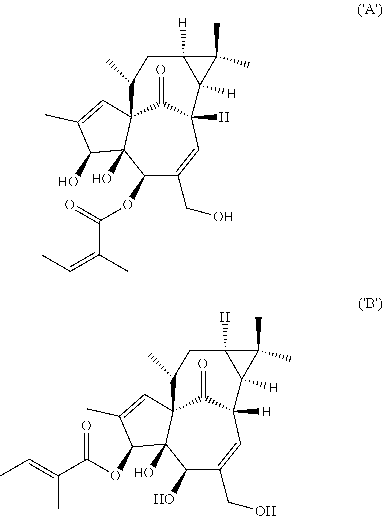

In some embodiments, where the composition does not fulfill the above criteria for chemical stable, the composition does exhibit a markedly improved stability over prior art hydrogel (Picato®). In particular these compositions preferably include gels where ingenol-3-angelate does not degrade by more than 15% when the gel is stored for 2 years at room temperature and/or the gels contain less than 12% of ‘A’ and/or 13′ by weight of total ingenanes. These values can be approximated as described above for the chemical stable gels.

In some embodiments, the composition is physically stable, where physically stable (or physical stability) means that the composition retains its macroscopic and microscopic appearance over the shelf-life of the product, e.g. any dissolved ingenol-3-angelate does not precipitate from the solvent phase.

In some embodiments, the composition is chemically stable and physically stable.

The inventors have found that a number of the compositions of the invention exhibit very favorable stability properties.

### Penetration and Permeation of the Compositions

The inventors have found that compositions of the invention can exhibit very favorable skin penetration characteristics. Skin penetration means the flux of the active ingredient into the different layers of the skin, i.e. the stratum corneum, epidermis and dermis, after application of the gel to the skin.

In some embodiments, the compositions exhibit greater flux, according to the in vitro diffusion test, of ingenol-3-angelate into the stratum corneum, epidermis and dermis after application of the gel to skin than does a reference gel of ingenol-3-angelate; wherein the reference gel (a) has the same strength of ingenol-3-angelate as the topical gel composition, (b) consists essentially of ingenol-3-angelate, benzyl alcohol, isopropyl alcohol in an amount of 30% by weight of the formulation, hydroxyethyl cellulose in an amount of 1.5% by weight of the formulation and citrate buffer solution in an amount of 67.55% by weight of the formulation, and (c) is prepared by mixing ingenol-3-angelate with benzyl alcohol, and then adding the remaining components to the mixture of ingenol-3-angelate and benzyl alcohol in the order of: isopropyl alcohol, a citrate buffer solution formed from citric acid in an amount of 0.56% by weight of the formulation, sodium citrate dihydrate in an amount of 0.14% by weight of the formulation and water in an amount of 66.85% by weight of the formulation, and then hydroxyethyl cellulose to form the reference gel.

If the total amount of ingenol-3-angelate in the stratum corneum, epidermis and dermis as a percentage of the applied dose, as determined in step (h), is higher than for the reference gel (e.g. PICATO® at the same strength of ingenol-3-angelate as the topical gel composition), then the composition is said to exhibit more penetration (i.e. greater flux of the active ingredient into the stratum corneum, epidermis and dermis after application of the gel to the skin).

In some embodiments, the composition exhibits less penetration than the reference gel according to this assay, i.e. the total amount of ingenol-3-angelate in the stratum corneum, epidermis and dermis (combined) as a percentage of the applied dose, as determined in step (h), is lower than for the reference gel (e.g. PICATO® at the same strength of ingenol-3-angelate as the topical gel composition).

Skin permeation means the flux of the active ingredient through the skin into the systemic circulation or, in case of in vitro studies, the receptor fluid of the Franz cell apparatus used in the experiment, after application of the gel to the skin. In some embodiments, the composition exhibits less permeation than does the reference gel according to this assay, where less potent permeation means that the amount of ingenol-3-angelate in the receptor fluid as a percentage of the applied dose, as determined in step (h), is lower than for the reference gel (e.g. PICATO® at the same strength of ingenol-3-angelate as the topical gel composition). This may be desirable to avoid unnecessary levels of systemic ingenol-3-angelate.

### Medical Treatments and Uses

The invention also provides a method for treating a dermal disease or condition, comprising topical administration of a gel of the invention to a mammal. Topical administration means that the compositions are applied cutaneously i.e. to the external skin on the body.

The invention also provides a gel of the invention for use in treating a dermal disease or condition.

The invention also provides the use of ingenol-3-angelate and a non-aqueous carrier in the manufacture of a gel medicament for treating a dermal disease or condition.

The uses and methods are useful for the topical treatment of dermal diseases or conditions including actinic keratosis, seborrheic keratosis, skin cancer, warts, keloids, scars, photoaged or photodamaged skin, and acne. In particular, the uses and methods are particularly useful for the topical treatment of actinic keratosis. The uses and methods may, for instance, be useful for the topical treatment of hyperkeratotic actinic keratosis.

The uses and methods may be used for the topical treatment of skin cancers such as non-melanoma skin cancer, malignant melanoma, Merkel cell carcinoma, squamous cell carcinoma or basal cell carcinoma (including superficial basal cell carcinoma and nodular basal cell carcinoma).

The uses and methods may be used for the topical treatment of warts, e.g. human papilloma virus (HPV) infections on the skin, genitals and mouth.

The uses and methods may be used for the topical treatment of photodamaged skin such as fine lines, wrinkles and UV-ageing. UV-ageing is often manifested by an increase in the epidermal thickness or epidermal atrophy, most notably by solar elastosis, the accumulation of elastin containing material just below the dermal-epidermal junction. Collagen and elastic fibres become fragmented and disorganised. At a cosmetic level this can be observed as a reddening and/or thickening of the skin resulting in a leathery appearance, skin fragility and irregular pigmentation, loss of tone and elasticity, as well as wrinkling, dryness, sunspots and deep furrow formation.

The uses and methods may be useful for reducing or minimizing scar tissue or improving cosmesis or functional outcome in a wound. For instance, the uses and methods may be useful for improving functional outcome in a wound which is cutaneous, chronic or diabetes associated, e.g. when the wound includes cuts and lacerations, surgical incisions, punctures, graces, scratches, compression wounds, abrasions, friction wounds, chronic wounds, ulcers, thermal effect wounds, chemical wounds, wounds resulting from pathogenic infections, skin graft/transplant, immune response conditions, oral wounds, stomach or intestinal wounds, damaged cartilage or bone, amputation sides and corneal lesions.

Therefore, in some embodiments, the uses and methods are cosmetic.

Typically, the uses and methods are lesion specific, i.e. they are focused on a lesion being treated and do not extend to any larger degree to the surrounding skin. In other embodiments, however, the uses and methods can extend to a larger area than the lesions, and this can usefully lead to treatment of emerging lesions or sub-surface pre-lesions. Also, it can be convenient to apply a gel to an area which includes several lesions, rather than applying it to each individual lesion in that area. The lesions could be of any size (i.e. surface area), e.g. greater than about 30 000 mm², greater than about 20 000 mm², greater than about 10 000 mm², greater than about 5000 mm², greater than about 1000 mm², greater than about 500 mm², greater than about 250 mm², or greater than about 150 mm². Typically, the lesion size is about 30 000 mm², about 20 000 mm², about 10 000 mm², about 5000 mm², about 1000 mm², about 500 mm², about 250 mm², about 150 mm², about 100 mm², about 75 mm², about 50 mm², about 25 mm² or about 10 mm².

In the treatment of, for example, actinic keratosis on the face and/or scalp of a subject, a gel composition of the invention may be applied on the face and scalp to the affected skin area (treatment area) once a day for 3 consecutive days. In the treatment of, for example, actinic keratosis on the trunk and/or extremities of a subject, a gel composition of the invention may be applied on the trunk and extremities to the affected skin area (treatment area) once a day for 2 consecutive days. Immediately following application of a gel to the treatment area, subjects should wash their hands.

The gel compositions of the invention are typically packaged in hermetically sealed containers, e.g. a unit dose tube. A unit dose tube would typically contain about 0.5 g of gel. Preferably, one unit dose tube (tube with screw cap or individual packets) may be used for one treatment area.

### MODES FOR CARRYING OUT THE INVENTION

The invention is further illustrated by the followings examples. It will be appreciated that the examples are for illustrative purposes only and are not intended to limit the invention as described above. Modification of detail may be made without departing from the scope of the invention.

### Example A

### Preparation of Compositions of the Invention

The following compositions were prepared:

Composition series 14

### 02A

PEP005 0.5 mg/g

Lipoid S PC (soy bean phosphatidylcholine) (phospholipid) 100 mg/g

Isopropanol 100 mg/g

Sepineo™ P600 25 mg/g

Citrate buffer pH 3.0 764.5 mg/g

Benzyl alcohol 10 mg/g

Prepared as follows: Dissolve Lipoid S PC in isopropanol without heating and under stirring. Add PEP005 to this solution and mix further, without heating. Add half of the total amount of buffer to this mixture and homogenize using a Silverson machine at 2-3000 rpm for two minutes. Solubilize Sepineo™ P600 in the remaining buffer solution and mix manually. Add the Sepineo™ P600 and buffer mixture to the Lipoid SPC, isopropanol, PEP005 and buffer solution mixture, and homogenize using a Silverson machine at 1500 rpm for five minutes. Finally, add benzyl alcohol and mix manually using a whisk.

### 08A

PEP005 0.5 mg/g

NanoSolve™ 100 mg/g (mixtures and solutions of phospholipids, polyols, carbohydrates and lipids)

Sepineo™ P600 30 mg/g

Citrate buffer pH 3.0 859.5 mg/g

Benzyl alcohol 10 mg/g

Prepared as follows: Solubilize NanoSolve™ in half of the total buffer solution and mix under stirring at 290 rpm for 30 minutes. Add PEP005 and mix for an hour without heating. Add benzyl alcohol and mix for 30 minutes. Solubilize Sepineo™ P600 in the remaining buffer solution and mix manually. Add the Sepineo™ P600 and buffer solution mixture to the NanoSolve™, buffer solution, PEP005 and benzyl alcohol mixture and mix until homogeneous.

### Composition Series 20

### 01A

PEP005 0.5 mg/g

NanoSolve™ 100 mg/g (mixtures and solutions of phospholipids, polyols, carbohydrates and lipids)

Hydroxyethyl cellulose HX (Natrosol® 250 HX) 15 mg/g

Citrate buffer 874.5 mg/g

Benzyl alcohol 10 mg/g

### Composition Series 62 (Niosomes)

### 03A

PEP005 0.5 mg/g

Ethyl alcohol 160 mg/g

Tween 80 50 mg/g (polysorbate)

Span 80 50 mg/g (Sorbitan monooleate)

Citrate buffer ad 1000

Sepineo™ P600 35 mg/g

### 04A

PEP005 0.5 mg/g

Ethyl alcohol 160 mg/g

Tween 80 50 mg/g (polysorbate)

Span 80 50 mg/g (Sorbitan monooleate)

Citrate buffer ad 1000

Hydroxyethyl cellulose 15 mg/g

### Niosome Preparation

The buffer is prepared. Divide the buffer into two parts. The lipid surfactants and API are dissolved in the alcohol. One part of the buffer and the alcoholic solution are added under constant mixing. The dispersion is homogenised by high pressure homogenisation.

### Final Formulation

The other part of buffer is used for the hydrogel preparation.

Disperse the gelling agent in the buffer.

When homogeneous hydrogel is obtained add the Niosome dispersion under homogenization.

### Composition Series 63

### 01A (Ethosomes)

PEP005 0.5 mg/g

Ethyl alcohol 300 mg/g Lipoid 100 20 mg/g (Phosphatidylcholine)

Citrate buffer pH 2.7 649.5

Natrosol 20 mg/g (hydroxyethylcellulose)

α-tocopherol 10 mg/g

### 02A (Ethosomes)

PEP005 0.5 mg/g

Isopropyl alcohol 300 mg/g

Lipoid 100 20 mg/g (Phosphatidylcholine)

Citrate buffer pH 2.7 649.5

Natrosol 20 mg/g (hydroxyethylcellulose)

α-tocopherol 10 mg/g

### 03A

PEP005 0.5 mg/g

Ethyl alcohol 300 mg/g

Citrate buffer pH 2.7 669.5

Natrosol 20 mg/g (hydroxyethylcellulose)

α-tocopherol 10 mg/g

### 04A

PEP005 0.5 mg/g

Isopropyl alcohol 300 mg/g

Citrate buffer pH 2.7 669.5

Natrosol 20 mg/g (hydroxyethylcellulose)

α-tocopherol 10 mg/g

### Preparation of Formulations:

Manufacture 1 L 1.5% Citrate buffer pH 2.7. Set 100 ml buffer to 30° C. in water bath. To redcap flask with magnetic stirring bar add 2% (w/w) Lipoid 100. Add Alcohol 30% (w/w). Add 1% (w/w) alpha-tocopherol. Close bottle with redcap flask and put tape or parafilm over cap hole. Dissolve by magnetic stirring 700 rpm at RT or already now set temp to 30° C. Add Ingenol mebutate in dry form (about 50 mg). Stir until dissolved. Add buffer slowly in a small gentle stream through the second hole in the redcap. Apply 700 rpm stirring during the entire time off addition of buffer. Once all buffer is added, remove dispenser and temperature probe and change to new red cap without holes. Continue mix for 5 minutes after buffer has been added. Add 2% (w/w) Natrosol. Turn off heat of magnetic stirring and reduce stirring to 100 rpm and let formulation cool to RT. Store final formulation in fridge (Cold formulation before filling. When filling 4 ml glass bottles, put on screw cap immediately, all in order to prevent evaporation)

### Composition Series 72 (Niosomes)

05A (Brij™ O5 niosome)

PEP005 0.5 mg/g

Benzylalcohol 10 mg/g

Isopropyl alcohol 50 mg/g

Brij™ O5 100 mg/g (ethoxylated natural fatty alcohol, based on oleyl alcohol)

0.2% Citrate buffer pH 3 ad 1000

### 07A (Span 83/EO Niosome 6/4)

PEP005 0.5 mg/g

Benzylalcohol 10 mg/g

Isopropyl alcohol 160 mg/g

Sorbitansesquioleate 60 mg/g

Ethyloleate 40 mg/g

0.2% Citrate buffer pH 3 ad 1000

Sepineo™ P600 35 mg/g

### 08A (Eithrol PG3PR/EO Niosome 6/4)

PEP005 0.5 mg/g

Benzylalcohol 10 mg/g

Isopropyl alcohol 100 mg/g

Cithrol™ PG3PR 60 mg/g (Polyglyceryl-3 Polyricinoleate)

Ethyloleate 40 mg/g

0.2% Citrate buffer pH 3 ad 1000

Sepineo™ P600 35 mg/g

### Niosome Preparation

The buffer is prepared. Divide the buffer into two parts (600 mg/g and the rest). The lipid surfactants and are dissolved in the alcohol, and then the benzylalcohol with or without API is added and mixed until dissolution. One part of the buffer (600 mg/g) is added slowly to the alcoholic solution under constant mixing (Silversson 3000-4500 rpm). Afterwards the dispersion is homogenised by high pressure homogenisation 3×5 min 500-800 Bar.

### Final Formulation

The rest of buffer is added to the gelling agent.

Disperse the gelling agent in the buffer by Silverson applying low shear rate.

Then add the Niosome dispersion under further homogenization.

Adjust pH to 3.5.

### Composition Series 81 (Procentages are Weight/Weight)

1

PEP005 0.05%

Phospholipon® 90H 0.59% (Phosphatidylcholine, hydrogenated)

Cholesterol 0.05%

2-hydroxyethylcellulose 1.4%

Citric acid 0.19%

1N NaOH or 1N HCl to pH 4.5

MilliQ water ad 100

2

PEP005 0.05%

Phospholipon® 90G 0.76%

Isopropanol 5.0 (volume/volume)

2-hydroxyethylcellulose 1.4%

Citric acid 0.19%

1N NaOH or 1N HCl to pH 4.5

water ad 100

3

PEP005 0.05%

Phospholipon®90G 0.76%

Isopropanol 5.0 (volume/volume)

2-hydroxyethylcellulose 1.4%

Citric acid 0.19%

1N NaOH or 1N HCl to pH 6.5

water ad 100

### Preparation of Liposomal Dispersions:

### Formulation 1:

Phospholipon® 90H (442.5 mg), cholesterol (37.5 mg) and PEP005 (37.5 mg) are dissolved in 20 ml chloroform and evaporated to dryness in a round-bottomed flask at room temperature at reduced pressure (60 mbar). The formed lipid film is hydrated with 75 ml citrate buffer pH 4.5 by rotation on a rotavapor at 40° C. for 50 minutes and 65° C. for 10 minutes. To help loosen the lipid film, the flask is ultrasonicated and agitated on a whirlmixer during the hydration. The resulting coarse dispersion is left to anneal for one hour at room temperature. After annealing, the dispersion is ultrasonicated in seven intervals of two minutes (7×2 min) over 50 minutes at 65° C. Immediately following dispersion the particle size distribution is determined by DLS.

### Formulation 2 and 3:

Phospholipon®90G (570 mg) and PEP005 (37.5 mg) are dissolved in 3.75 ml isopropanol and the resulting solution is dispersed into 71.25 ml citrate buffer. The dispersion is ultrasonicated by probe in seven intervals of two minutes (7×2 min) at room temperature. Immediately following dispersion the particle size distribution is determined by DLS.

### Thickening of Prepared Liposomal Preparations:

The liposome dispersions are thickened by addition of 1050 mg 2-hydroxyethylcellulose to the liposomal preparations. The added 2-hydroxyethylcellulose is hydrated by stirring the preparations for two hours at room temperature followed by over-night storage at 5° C., during which the hydration is completed.

### Examples B

### Stability Studies

A number of compositions of the invention were tested for chemical stability. This testing required extraction of ingenol-3-angelate from the composition by dissolution in a solvent mixture of acetonitrile and phosphoric acid. Following extraction, organic impurities were identified using reversed phase HPLC with UV detection at 220 nm. The following composition from Example A was found to be stable after 6 months at 25° C., or 3 months at 40° C. indicating that less than 10% of the ingenol-3-angelate would be expected to degrade over a storage period of 2 years at room temperature (25° C.) and/or that the formulations were expected to contain less than 5% by weight of total ingenanes of the degradation products ‘A’ and/or ‘B’:

Compositions series 63, formulations 01 and 02.

The following compositions were found to have a markedly improved stability over prior art hydrogel (Picato®), indicating ingenol-3-angelate was not expected to degrade by more than 15% when the gel is stored for 2 years at room temperature and/or the gels were expected to contain less than 12% of ‘A’ and/or 13′ by weight of total ingenanes:
Composition 14, formulation 08A
Composition 62, formulation 03A

Composition from series 81, formulations 1 and 2 showed much improved stability over Picato® gel with a similar pH value (i.e. improved stability of ingenol-3-angelate when compared to corresponding isopropanol gels with a higher pH than Picato® (which is also an isopropanol gel)). The compositions may not reach full 2 years stability at room temperature.

Composition from series 81, formulations 1 and 2 showed much improved stability over Picato® gel with a similar pH value (i.e. improved stability of ingenol-3-angelate when compared to corresponding isopropanol gels with a higher pH than Picato® (which is also an isopropanol gel)). The compositions may not reach full 2 years stability at room temperature.

### Example C

### Skin Penetration and Permeation Studies

To investigate the skin penetration and permeation of ingenol-3-angelate from compositions of the invention, an in vitro skin diffusion test was conducted.

Full thickness skin from pig ears was used in the study. The ears were kept frozen at −18° C. before use. On the day prior to the experiment the ears were placed in a refrigerator (5±3° C.) for slow defrosting. On the day of the experiment, the hairs were removed using a veterinary hair trimmer. The skin was cleaned for subcutaneous fat using a scalpel and two pieces of skin were cut from each ear and mounted on Franz diffusion cells in a balanced order.

Flow-through Franz-type diffusion cells with an available diffusion area of 3.14 cm² and receptor volumes ranging from 11.1 to 12.6 ml were used in substantially the manner described by T. J. Franz, “The finite dose technique as a valid in vitro model for the study of percutaneous absorption in man”, in Current Problems in Dermatology, 1978, J.W.H. Mall (Ed.), Karger, Basel, pp. 58-68. The specific volume was measured and registered for each cell. A magnetic bar was placed in the receptor compartment of each cell. After mounting the skin, physiological saline (35° C.) was filled into each receptor chamber for hydration of the skin. The cells were placed in a thermally controlled water bath which was placed on a magnetic stirrer set at 400 rpm. The circulating water in the water baths was kept at 35±1° C. resulting in a temperature of about 32° C. on the skin surface. After half an hour the saline was replaced by receptor medium, 0.04 M isotonic phosphate buffer, pH 7.4 (35° C.), containing 4% bovine serum albumin and left for hydration another hour. The inlet and outlet ports of the receptor chamber were connected to stainless steel HPLC tubing. The cells were connected to a 12-channel peristaltic pump, and the receptor fluid was pumped continuously through each cell and collected in vials placed at a fraction collector. A controller was used to program independently the duration of each fraction. Sink conditions were maintained at all times during the period of the study, i.e. the concentration of the active compounds in the receptor medium was below 10% of the solubility of the compounds in the medium.

The in vitro skin penetration and permeation was tested in 6 replicates (i.e. n=6). Each test composition was applied to the skin membrane at 0 hours in an intended dose of 4 mg/cm². A glass spatula was used for the application, and the residual amount of the composition was determined so as to give the amount of the composition actually applied on the skin.

### Permeation and Penetration

The skin penetration and permeation experiment was allowed to proceed for 21 hours. Samples were then collected from the following compartments:

About 6 ml of the receptor fluid was sampled from each cell every third hour until 21 hours post application. The sample collection of the first 45 minutes was discarded due to the lag time of the system. The recipient fluid remaining in the diffusion cell at the end of the study corresponded to the 21 hour sample.

The stratum corneum was collected by tape stripping 10 times using D-Squame® tape (diameter 22 mm, CuDerm Corp., Dallas, Tex., USA). Each tape strip was applied to the test area using a standard pressure for 5 seconds and removed from the test area in one gentle, continuous move. For each repeated strip, the direction of tearing off was varied. The viable epidermis and dermis was then sampled from the skin by taking a full biopsy of 3.14 cm² of the applied area for analysis. The skin surrounding the test area was discarded.

The concentration of ingenol-3-angelate in the samples was determined by LC-MS/MS.

### Results

These studies allowed the amount of ingenol-3-angelate found in the stratum corneum, epidermis and dermis and receptor fluid to be calculated, as a percentage of the applied dose.

The following composition from Example A exhibited more penetration than reference gel at the same strength of ingenol-3-angelate by weight of the composition:
Composition series 14, formulation 08A

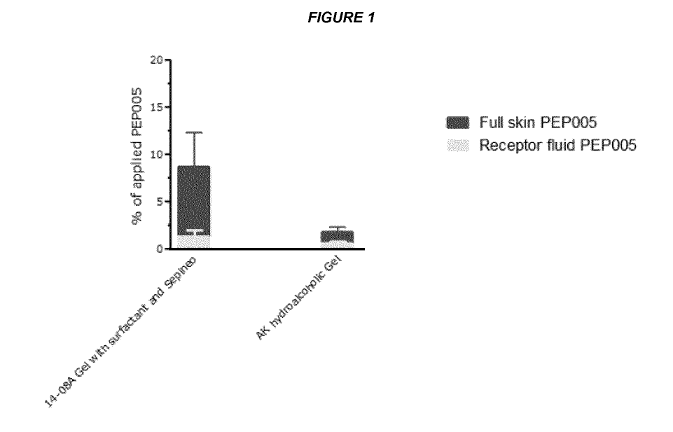

The data for composition series 14, formulation 08A are shown in FIG. 1, which show that the amount of ingenol-3-angelate found in the stratum corneum, epidermis and dermis after application of the composition is significantly higher than the amount found in the stratum corneum, epidermis and dermis after application of PICATO® at the same strength of ingenol-3-angelate by weight of the composition.

It will be understood that the invention has been described by way of example only and modifications may be made whilst remaining within the scope and spirit of the invention.

1. An aqueous topical liposome gel composition comprising ingenol-3-angelate.

2. The composition of claim 1, wherein the ingenol-3-angelate is present in an amount of about 0.0005%, about 0.001%, about 0.0025%, about 0.005%, about 0.01%, about 0.015%, about 0.025%, about 0.05%, about 0.075%, about 0.1%, about 0.125%, about 0.15%, about 0.2%, about 0.25% or about 0.5% by weight of the composition.

3. The composition of claim 1, wherein the ingenol-3-angelate is present in an amount of about 0.015% or about 0.05% by weight of the composition.

4. The composition of claim 1, wherein the aqueous topical liposome gel composition includes a vesicular systems, wherein the vesicular system is formed from one or more naturally occurring or synthetic lipid compounds or surfactants, or a mixture thereof.

5. The composition of claim 4, wherein the vesicular systems comprises a phospholipid.

6. The composition of claim 5, wherein the phospholipid is selected from soybean lecithin, egg lecithin, lecithin, lysolecithin, phosphatidylserine, phosphatidylethanolamine, phosphatidylcholine and phosphatidylinositol, phosphatidylglycerol and phosphatidylacid.

7. The composition of claim 5, wherein the phospholipid is mixed with a sterol.

8. The composition of claim 7, wherein the sterol is cholesterol.

9. The composition of claim 4, wherein the vesicular systems comprises a non-phosphorous-containing lipid or surfactant or a mixture thereof.

10. The composition of claim 4, wherein the lipid is chemically or physically modified.

11. The composition of claim 4, wherein the lipid is present in an amount of from about 0.1% to about 98% by weight of the composition.

12. The composition of claim 1, wherein the composition is acidic.

13. The composition of claim 1, wherein the composition has a pH of less than about 4.5.

14. The composition of claim 1, wherein the composition includes an aqueous buffer solution.

15. The composition of claim 14, wherein the composition includes from about 2.5% to about 99.9% buffer solution by weight of the composition.

16. The composition of claim 14, wherein the composition includes a citrate buffer.

17. The composition of claim 1, wherein the composition includes an emulsifier.

18. The composition of claim 17, wherein the viscosity-increasing ingredient is present in an amount of from about 1% to about 20% by weight of the composition.

19. The composition of claim 1, wherein the composition includes a non-aqueous carrier.

20. The composition of claim 19, wherein the non-aqueous carrier is present in an amount of from about 1% to about 40% by weight of the composition.

21. The composition of claim 1, wherein the composition includes a viscosity-increasing ingredient.

22. The composition of claim 21, wherein the viscosity-increasing ingredient is present in an amount of from about 0.5% to about 40% by weight of the composition.

23. The composition of claim 1, wherein the composition includes a co-solvent.

24. The composition of claim 23, wherein the co-solvent is present in an amount of from about 0.5% to about 40% by weight of the composition.

25. The composition of claim 1, wherein the composition includes a penetration enhancer.

26. The composition of claim 25, wherein the composition includes from about 0.01% to about 20% by weight of the composition.

27. The composition of claim 1, wherein the composition includes an acidifying compound.

28. The composition of claim 27, wherein the acidifying compound is present in an amount of from about 0.5% to about 10% by weight of the composition.

29. The composition of claim 1, wherein the composition includes a silicone.

30. The composition of claim 29, wherein the silicone functions as a non-aqueous carrier or a viscosity-increasing ingredient.

31. The composition of claim 1, wherein the composition is chemically stable.

32. The composition of claim 1, wherein the composition is physically stable.

33. The composition of claim 1, wherein the composition exhibits more penetration than a reference gel of the same strength of ingenol-3-angelate according to an in vitro diffusion test, wherein the reference gel has (a) same strength of ingenol-3-angelate as the composition, (b) consists essentially of ingenol-3-angelate, benzyl alcohol, isopropyl alcohol in an amount of 30% by weight of the formulation, hydroxyethyl cellulose in an amount of 1.5% by weight of the formulation and citrate buffer solution in an amount of 67.55% by weight of the formulation, and (c) is prepared by mixing ingenol-3-angelate with benzyl alcohol, and then adding to the mixture of ingenol-3-angelate and benzyl alcohol in order of: isopropyl alcohol, a citrate buffer solution formed from citric acid in an amount of 0.56% by weight of the formulation, sodium citrate dihydrate in an amount of 0.14% by weight of the formulation and water in an amount of 66.85% by weight of the formulation, and then hydroxyethyl cellulose to form the reference gel.

34. The composition of claim 1, wherein the composition exhibits less permeation than a reference gel of the same strength of ingenol-3-angelate, according to an in vitro diffusion test, wherein the reference gel has (a) same strength of ingenol-3-angelate as the composition, (b) consists essentially of ingenol-3-angelate, benzyl alcohol, isopropyl alcohol in an amount of 30% by weight of the formulation, hydroxyethyl cellulose in an amount of 1.5% by weight of the formulation and citrate buffer solution in an amount of 67.55% by weight of the formulation, and (c) is prepared by mixing ingenol-3-angelate with benzyl alcohol, and then adding to the mixture of ingenol-3-angelate and benzyl alcohol in order of: isopropyl alcohol, a citrate buffer solution formed from citric acid in an amount of 0.56% by weight of the formulation, sodium citrate dihydrate in an amount of 0.14% by weight of the formulation and water in an amount of 66.85% by weight of the formulation, and then hydroxyethyl cellulose to form the reference gel.

35. A method for making a composition of claim 1, comprising mixing ingenol-3-angelate with one or more naturally occurring or synthetic lipid compounds.

36. The method of claim 35, comprising mixing ingenol-3-angelate with a solvent and one or more naturally occurring or synthetic lipid compounds, followed by drying under vacuum evaporation to yield a dry lipid film, followed by mixing the film with an aqueous buffer solution and then homogenizing the mixture using a dispersion process.

37. The method of claim 35, comprising mixing ingenol-3-angelate with a solvent and one or more naturally occurring or synthetic lipid compounds, followed by mixing the lipid mixture with an aqueous buffer solution and then homogenizing the mixture using a dispersion process.

38. A method for treating a dermal disease or condition, comprising topical administration of a composition of claim 1 to a mammal.

39. The method of claim 38, wherein the dermal disease or condition is actinic keratosis.

40. The composition of claim 6, wherein the phospholipid is mixed with a sterol.

41. The composition of any one of claims 5 to 10, wherein the lipid is present in an amount of from about 0.1% to about 98% by weight of the composition.

42. The composition of claim 6, wherein the lipid is present in an amount of from about 0.1% to about 98% by weight of the composition.

43. The composition of claim 7, wherein the lipid is present in an amount of from about 0.1% to about 98% by weight of the composition.

44. The composition of claim 8, wherein the lipid is present in an amount of from about 0.1% to about 98% by weight of the composition.

45. The composition of claim 9, wherein the lipid is present in an amount of from about 0.1% to about 98% by weight of the composition.

46. The composition of claim 10, wherein the lipid is present in an amount of from about 0.1% to about 98% by weight of the composition.

47. A method for treating a dermal disease or condition, comprising topical administration of a composition of any preceding claim 5 to a mammal.

48. The method of claim 47, wherein the dermal disease or condition is actinic keratosis.

49. A method for treating a dermal disease or condition, comprising topical administration of a composition of any preceding claim 35 to a mammal.

50. The method of claim 49, wherein the dermal disease or condition is actinic keratosis.

51. A method for treating a dermal disease or condition, comprising topical administration of a composition of any preceding claim 36 to a mammal.

52. The method of claim 51, wherein the dermal disease or condition is actinic keratosis.

53. A method for treating a dermal disease or condition, comprising topical administration of a composition of any preceding claim 37 to a mammal.

54. The method of claim 53, wherein the dermal disease or condition is actinic keratosis.
